SEQUENCE LISTING (5 sequences)

SEQ ID NO: 1 - protein, 12 aa, Artificial
  Synthetic Construct; Antigen
SSVLYGGPPSAA

SEQ ID NO: 2 - protein, 12 aa, Artificial
  Synthetic Construct; Antigen
NYQTYTPRPPHS

SEQ ID NO: 3 - protein, 12 aa, Artificial
  Synthetic Construct; Antigen
VTNNQTSPRWEI

SEQ ID NO: 4 - protein, 12 aa, Artificial
  Synthetic Construct; Antigen
WKPVSLTLHTHP

SEQ ID NO: 5 - protein, 12 aa, Artificial
  Synthetic Construct; Antigen
HATGTHGLSLSH

(19) United States
(12) Patent Application Publication
(10) Pub. No.: US 2009/0081247 A1
(43) Pub. Date: Mar. 26, 2009

(54) Immune adjuvant comprising ATP

(76) Inventors: Shuji Sato, Narita-Shi (JP); Takeshi Goto, Ushiku-Shi (JP); Naoya Ohmori, Chiba-Shi (JP); Kueichen Chiang, Tokyo-To (JP); Yayoi Shimada, Kisarazu-Shi (JP)

Correspondence Address: WENDEROTH, LIND & PONACK, L.L.P., 2033 K STREET N. W., SUITE 800, WASHINGTON, DC, 20006-1021, US

(21) Appl. No.: 12/213,874

(22) Filed: Jun. 25, 2008

Related U.S. Application Data
(60) Provisional application No. 60/929,404, filed on Jun. 26, 2007.

(51) Int. Cl. A61K 39/02; A61K 39/00; A61K 47/06; A61P 37/00; A61P 37/06

(52) U.S. Cl. 424/185.1; 424/278.1; 424/184.1; 424/204.1; 424/234.1; 424/277.1

(57) ABSTRACT

This invention relates to an immunoadjuvant, which has an excellent antibody production enhancing function and is highly safe, and a vaccine composition comprising the immunoadjuvant. More specifically, the present invention relates to an immunoadjuvant comprising ATP or its pharmaceutically acceptable salt, its solvate, or its derivative having a physiological function as an active ingredient, and a vaccine composition comprising the immunoadjuvant.

### BACKGROUND OF THE INVENTION

1. Field of the Invention

The present invention relates to an immunoadjuvant comprising ATP or its pharmaceutically acceptable salt, its solvate, or its derivative, and a vaccine composition comprising the immunoadjuvant.

2. Background Art

Antigenic substances such as extraneous proteins and polysaccharides are known to be inoculated as a vaccine into organisms in the treatment or prevention of infectious diseases and the like. However, the amount of the antibody produced by the organism and induced by antigenic substances is sometimes disadvantageously unsatisfactory in view of the defense of the organism against diseases.

The development of an immunoadjuvant which is administered to an organism together with an antigenic substance has hitherto been carried out with a view to enhancing the immunogenicity of vaccines.

Conventional immunoadjuvants include, for example, Freund adjuvants, aluminium salts (Alm), virosomes, exotoxins, MF 59, saponins, LPSs, cytokines, and CpG oligonucleotides (Expert. Rev. Vaccine, Vol. 2 (2), 167-188 (2003)). These conventional immunoadjuvants, however, cause grave side effects or is unsatisfactory in immunopotentiating action, and has a limitation in diseases to which the immunoadjuvant can be applied.

Dermal vaccines are recognized as significantly increasing the production of an IgG antibody in the blood and as useful in the treatment or prevention of infectious diseases and the like. The defending ability of the dermal vaccine in a membrana mucosa which is an invasion port of pathogens, however, is generally low. Thus, adjuvants for dermal administration are required for compensating for the low defending ability. For example, cholera toxins are reported as a conventional adjuvant suitable for dermal administration (Vaccine, Vol. 23, 2511-2519 (2005), Vaccine, Vol. 24, 6110-6119 (2006)). The cholera toxins have an adjuvant effect in animal experiments, but on the other hand, any adjuvant effect, which can induce immunoresponce on a satisfactory level, is not observed in clinical trials.

Accordingly, the development of an excellent novel immunoadjuvant, which has the function of effectively increasing antibody production in organisms, and a vaccine composition using the immunoadjuvant have still been desired.

### SUMMARY OF THE INVENTION

The present inventors have now found that ATP can be used as an excellent immunoadjuvant having the function of effectively enhancing the production of an antibody against antigenic substances.

The present invention has been made based on such finding.

Accordingly, an object of the present invention is to provide an excellent novel immunoadjuvant, which can effectively enhance antibody production, and a vaccine composition comprising the immunoadjuvant.

The immunoadjuvant according to the present invention is characterized by comprising ATP or its pharmaceutically acceptable salt, its solvate, or its derivative having a physiological function.

Further, the vaccine composition according to the present invention is characterized by comprising ATP or its pharmaceutically acceptable salt, its solvate, or its derivative having a physiological function, and an antigenic substance.

The immunoadjuvant according to the present invention has the function of significantly enhancing the production of an antibody against antigenic substances in vivo and can be advantageously utilized in the immunological treatment or prevention of various diseases. The immunoadjuvant according to the present invention comprises ATPs or the like as an active ingredient and thus can be advantageously utilized as safe immunoadjuvants against organisms.

### BRIEF DESCRIPTION OF THE DRAWINGS

FIG. 1 is a diagram showing the results of measurement of the amount of produced antibody by ELISA with the use of the immunoadjuvant according to the present invention. For reference, the results of measurement of the amount of produced antibody by ELISA with the use of cholera toxin or without the use of any immunoadjuvant.

FIG. 2 is a diagram showing IgG1/IgG2a values in blood samples with the use of the immunoadjuvant according to the present invention. For reference, IgG1/IgG2a values in blood samples with the use of cholera toxin or without the use of any immunoadjuvant.

### DETAILED DESCRIPTION OF THE INVENTION

### Definition

The term “immunoadjuvant” as used herein refers to a substance which, when administered together with an antigenic substance to organisms, can enhance immunoresponse to the antigenic substance.

The expression “derivative having a physiological function” as used herein refers to a chemical derivative of ATP without sacrificing the physiological function possessed by ATP and embraces, for example, compounds which are converted in vivo to produce ATP.

The expression “peptide having a functionally equivalent activity” as used herein refers to the following peptide.

It is known that, in peptides, polymorphisms or mutants of genes coding them are present, and, in addition, some peptides may cause mutations such as substitutions, deletions, additions or the like of amino acids in the amino acid sequence, for example, by modifications in vivo or during purification, or artificial manipulation but nevertheless exhibit physical and biological activities substantially equivalent to peptides having no mutation. Such peptides, which, even when there is the above structural difference, have a function substantially equivalent to peptides having no mutation, refer to “peptides having functionally equivalent activity”

The term “alkyl,” “alkoxy,” “alkenyl,” or “alkynyl” refers to a straight chain, branched chain, or cyclic alkyl, alkoxy, alkenyl, or alkynyl group.

The term “aryl” as used herein refers to phenyl or naphthyl unless otherwise specified. The term “heteroaryl” as used herein refers to a five or six-membered heteroaryl having one to three nitrogen, oxygen or sulfur atoms (a five- or six-membraned aromatic heterocyclic group) unless otherwise specified.

The term “treatment” as used herein means ameliorating an established disease state, and the term “prevention” as used herein means preventing the establishment of a disease state in the future.

The term “histonH1-like antigen” as used herein refers to an antigen recognized in cell membranes in splenocytes by monoclonal antibodies produced by hybridoma 1F5, hybridoma 3F2, hybridoma 15F11, hybridoma 17C2, or hybridoma 16G9. The above-described hybridoma 1F5, hybridoma 3F2, hybridoma 15F11, hybridoma 17C2, and hybridoma 16G9 have been deposited with International Patent Organism Depositary, National Institute of Advanced Industrial Science and Technology (address: Tsukuba Central 6 Tsukuba-shi, Higashi 1-1-1, Ibaraki, Japan) (original deposited date: Aug. 19, 2004) under accession number FERM BP-10409, accession number FERM BP-10410, accession number FERM BP-10411, accession number FERM BP-10412, and accession number FERM BP-10413, respectively.

Immunoadjuvant

As described above, one feature of the immunoadjuvant according to the present invention is that the immunoadjuvant comprises ATP (adenosine triphosphate) or its pharmaceutically acceptable salt, its solvate, or its derivative having a physiological function.

ATP is known as a nucleotide which participates in the conservation and utilization of energy used in vivo. It is a surprising fact that, when the ATP is used as an immunoadjuvant, humoral immunity in which Th2 cells are predominant are induced to effectively enhance antibody production. According to the present invention, the above ATP or its derivative can be used as an immunoadjuvant to effectively enhance antibody production.

The ATP in the present invention may be used in a salt form. Examples of such salts include pharmaceutically acceptable nontoxic salts. Examples of suitable salts include salts such as alkali metal salts (for example, sodium salts and potassium salts), alkaline earth metal salts (for example, calcium salts and magnesium salts), ammonium salts, and organic bases.

ATP may be used as its solvate. Preferred solvents are, for example, hydrates or organic solvate such as ethanolates.

Further, in the present invention, ATP derivatives having a physiological function may be used as an immunoadjuvant. Examples of suitable derivatives include esters or amides. Such esters or amides can be synthesized by a process known in the art.

The ester is preferably a compound in which one or more hydroxyl groups contained in ATP have been converted to ester groups. Examples of such preferred esters include carboxylate esters, sulfonate esters, and amino acid esters, for example, alkyl esters, alkenyl esters, alkynyl esters, alkoxyalkyl esters, heteroaryl esters, aryl esters, and aralkyl esters, and mono-, di-, or tri-phosphonate esters. In a further preferred embodiment of the present invention, the ester group is a group which can be converted to a hydroxyl group in vivo.

The amide is preferably a compound in which the amino group contained in ATP has been converted to an amide group. Examples of suitable amides include alkylamides, alkenylamides, alkynylamides, alkoxyalkylamies, heteroarylamide, arylamides, and aralkylamides. In a further preferred embodiment of the present invention, the amide group is a group which can be converted to an amino group in vivo.

In the ester or amide, it is advantageous that the alkyls, alkenyls and alkynyls each contain 1 to 6 carbon atoms, preferably 1 to 4 carbon atoms. It is further advantageous in that the aryls each contain a phenyl group.

One or more hydrogen atom contained in the ester or amide may be substituted, and examples of preferred substituents include a hydroxyl group and halogen atoms (for example, chlorine, bromine, or fluorine).

Among physiological functions of ATP derivatives, the immunoadjuvant activity can be confirmed by a method well known by a person having ordinary skill in the art. For example, the administration of an antigenic substance and an ATP derivative to an organism enhances the titer of the antibody against the antigenic substance. The immunoadjuvant activity of the ATP derivative can be confirmed by comparing the titer of the antibody with that when ATP is used as an immunoadjuvant.

The immunoadjuvant according to the present invention may contain other ingredients so far as the antibody production enhancing effect attained, for example, by ATP is not sacrificed. Such other ingredients include, for example, binders, colorants, desiccants, antiseptics, wetting agents, stabilizers, excipients, adhesives, plasticizers, tackifiers, thickeners, patch materials, ointment bases, keratin removers, basic substances, absorption promoters, fatty acids, fatty acid ester, higher alcohols, surfactants, water, and buffer agents. Preferred other ingredients include buffer agents, ointment bases, fatty acids, antiseptics, basic substances, or surfactants.

The content of ATP and the like in the immunoadjuvant according to the present invention may be properly determined by taking into consideration, for example, the properties of the antigenic substance used, the necessary amount of the antibody, and the dosage form and may be, for example, 1 to 100% by weight. The immunoadjuvant according to the present invention is produced by properly mixing ATP and the like and the above various ingredients together.

The above effect of ATP and the like as an immunoadjuvant is particularly advantageous when they, together with dermal vaccine, is utilized in the prevention or treatment of various diseases. Accordingly, the immunoadjuvant according to the present invention is preferably utilized as an adjuvant for dermal administration.

Vaccine Composition

The immunoadjuvant according to the present invention may be administered separately from the antigenic substance in the administration to organisms. Alternatively, the immunoadjuvant according to the present invention, together with the antigenic substance, can be administered as a vaccine composition.

The antigenic substance in the vaccine composition may be properly selected depending, for example, upon target diseases and the nature of patients and is not particularly limited so far as the antigenic substance, together with ATP or its derivative, induces immunoresponse. Examples of suitable antigenic substances include peptides, proteins (for example, glucoproteins and lipoproteins), carbohydrates (for example, polysaccharides), lipids (for example, glycolipids), nucleic acids (for example, oligonucleotides, single stranded DNAs, double stranded DNAs, RNAs, or plasmid DNAs), or toxoids. Preferred are peptides and proteins.

The antigenic substance may be a naturally occurring antigenic substance or may be an antigenic substance synthesized by a chemical process or a DNA recombinant technique. Such antigenic substances include, for example, virus derived antigens (for example, recombinant viruses, virus lysates, and virus analogues such as virosomes), bacteria-derived antigens (for example, bacteria lysates), and cancer relates antigens (for example, cancer cell lysates).

A plurality of types of antigenic substances may be used in combination as the antigenic substance, and the present invention embraces this embodiment. The vaccine composition according to the present invention can be used in the treatment or prevention of various diseases depending, for example, upon the type and properties of the antigenic substance. When the antigenic substance can induce the production of an antibody having immunosuppressive activity, the vaccine composition according to the present invention is advantageous in the prevention or treatment of transplant rejection in vivo, particularly organ transplantation patients. Accordingly, in another preferred embodiment of the present invention, the vaccine composition can be used in the prevention or treatment of transplant rejection.

In a preferred embodiment of the present invention, the antigenic substance comprises a peptide selected from the following peptides (a) and (b):

(a) a peptide having an amino acid sequence represented by SSVLYGGPPSAA (SEQ ID No. 1); and

(b) a peptide comprising an amino acid sequence represented by SSVLYGGPPSAA (SEQ ID No. 1) wherein one or a few amino acids have been substituted, deleted, or added, the polypeptide having an activity functionally equivalent to the peptide described in the item (a).

The antigenic substance is particularly advantageous in the induction of the production of an antibody having immunosuppressive activity in vivo.

In the peptide described in the above item (b), the expression “one or a few” refers to preferably approximately 1 to 3, more preferably approximately 1 or 2.

Whether or not the peptide described in the above item (b) has an activity which is functionally equivalent to the peptide described in the above item (a) can be confirmed by conventional assay methods, for example, a method in which the amount of an antibody produced by administering a peptide to an organism is measured, for example, by ELISA, or a method in which the immunosuppressive function of the antibody is compared by a mixed lymphocyte reaction (an MLR reaction). The above antigenic substances and assay methods thereof are described by the present inventors in WO 2006/205580 and are incorporated herein by reference.

In addition to the above peptides, examples of suitable antigenic substances, which can induce the production of an antibody having immunosuppressive activity, are described in WO 2006/205580. Specific examples of such antigenic substances include histone H1, histone H1-like antigen, peptides having amino acid sequences represented by NYQTYTPRPPHS (SEQ ID No. 2), VTNNQTSPRWEI (SEQ ID No. 3), WKPVSLTLHTHP (SEQ ID No. 4), or HATGTHGLSLSH (SEQ ID No. 5), peptide analogs having an activity functionally equivalent to the peptides, or complexes or mixtures comprising them. For example, peptide analogs having the same substitution, deletion, or addition as the peptide of the above item (b) may be mentioned as the above peptide analog.

In a preferred embodiment of the present invention, the vaccine composition further comprises a pharmaceutically acceptable carrier. When the antigenic substance has a low molecular weight, the administration of a complex of the carrier and the antigenic substance bound to each other to an organism is particularly advantageous for effectively inducing the immunoresponse. Accordingly, in a more preferred embodiment of the present invention, the carrier is bounded to the antigenic substance. Keyhole limpet hemocyanin (KLH), ovalbumin (OVA) or bovine serum albumin (BSA) are preferred carrier. KLH is more preferred.

In a further preferred embodiment of the present invention, the antigenic substance is a product of binding between the polypeptide described in the above item (a) or (b) and a carrier selected from KLH, OVA, or BSA. In a further preferred embodiment, the antigenic substance is a product of binding between the polypeptide described in the above item (a) or (b) and KLH. In another preferred embodiment of the present invention, the antigenic substance is a product of binding between histone H1 or histone H1-like antibody and a carrier selected from KLH, OVA, and BSA.

When the antigenic substance is artificially synthesized, for example, conventional peptide synthesis techniques such as peptide solid phase synthesis methods and peptide liquid phase synthesis methods may be used. The method for binding the antigenic substance to the carrier is not particularly limited so far as the immunogenicity of the antigenic substance is not sacrificed. For example, a method may be adopted in which an antigenic substance is bound to a carrier with dehydration condensing agents, for example, EDC (ethylene dichloride), DCC (dicyclohexyl carbodiimide), DIC (1,3-diisopropyl carbodiimide), crosslinking agents, for example, glutaraldehyde, maleimide, maleimidebenzoyloxysuccinic acid, PEG, and linkers, for example, linker peptides. In a preferred embodiment of the present invention, the antigenic substance and the carrier are bound to each other through carbodiimide or glutaraldehyde. Regarding the process for producing a product of binding between the peptide and the carrier, see the process described, for example, in Nobuo Izumiya et al., “Pepuchido Gosei No Kiso To Jikken (Basis and Experiments of Peptide Synthesis),” published by Maruzen Co., Ltd.

The vaccine composition according to the present invention may further comprise the above other ingredients. Examples of suitable other ingredients include superantigens, cytokines, cholera toxins or mutants thereof, heat-labile enterotoxins or mutants thereof, and CpG oligonucleotides. The addition of the above ingredients is advantageous for further enhancing the function of the antigenic substance as the immunogen.

The amount of the antigenic substance in the vaccine composition according to the present invention is not particularly limited so far as the amount is an immunologically effective amount to a target disease. The amount of the antigenic substance may be properly determined by a person having ordinary skill in the art such as physicians depending, for example, upon the age and weight of the organism and the properties and progress of diseases. The amount of the antigenic substance in the vaccine composition may be, for example, 1 to 50% by weight.

The amount of the immunoadjuvant in the vaccine composition may be properly determined by a person having ordinary skill in the art while taking into consideration the amount of the immunoadjuvant effective for enhancing an immunoreaction against the antigenic substance in the organism, using, for example, the amount of antibody produced in the organism as an index and may be, for example, 1 to 50% by weight.

Use

The above vaccine composition may be formulated by a method known in the art of formulation, for example, into liquid preparations, suspensions, ointments, powders, lotions, W/O emulsions, O/W emulsions, emulsions, creams, cataplasms, patches, and gels and is preferably used as medicaments. Thus, according to another aspect of the present invention, there is provided a pharmaceutical composition comprising the above vaccine composition. The vaccine composition according to the present invention, when dermally administered, can significantly induce antibody production. Accordingly, in another preferred embodiment of the present invention, the vaccine composition can be provided as a transdermal preparation.

Further, as described above, ATP or its derivative according to the present invention is administered, to an organism, together with the antigenic substance, as a vaccine composition, or as an immunoadjuvant which is a preparation separately from the antigenic substance, whereby the amount of an antibody produced in the organism can be significantly increased. Thus, according to a still another aspect of the present invention, there is provided a method for increasing the amount of an antibody produced against an antigenic substance in an organism, the method comprising administering an immunologically effective amount of the antigenic substance, and ATP or its pharmaceutically acceptable salt, its solvate, or its derivative having a physiological function in an amount effective as an immunoadjuvant simultaneously or successively into the organism.

When the immunoadjuvant according to the present invention and an antigenic substance, which can induce the production of an antibody having immunosuppressive activity, are administered to an organism, the transplant rejection can be effectively treated or prevented. Thus, according to a further aspect of the present invention, there is provided a method for inhibiting transplant rejection in organisms, the method comprising administering an immunologically effective amount of the above antigenic substance, and ATP or its pharmaceutically acceptable salt, its solvate, or its derivative having a physiological function in an amount effective as an immunoadjuvant simultaneously or successively into the organism. The antigenic substance in the above method is the same as the antigenic substance which can induce the production of an antibody having immunosuppressive activity in the vaccine composition.

The effective amount of the above ATP as an immunoadjuvant and the immunologically effective amount of the antigenic substance may be properly determined by a person having ordinary skill in the art by taking into consideration, for example, the type and properties of the antigenic substance, the species of organisms, age, body weight, severity of diseases, the type of diseases, the time of administration, and administration method and further using the amount of an antibody produced against the antigenic substance in the organism as an index.

The antigenic substance, immunoadjuvant, or vaccine composition according to the present invention can be administered to organisms by a suitable method selected depending, for example, upon the condition of patients and properties of diseases. Examples of such methods include intraperitoneal administration, dermal administration for example, subcutaneous injection, intradermal injection, and patching, nosal administration, oral administration, mucosa administration (for example, rectal administration, vaginal administration, and corneal administration). Among them, dermal administration is preferred. Other methods include a method in which, after mixing immunocompetent cells with an immunoadjuvant, an antigenic substance and the like in vitro, the mixture is administered to an organism to stimulate an immunoreaction in vivo. Such immunocompetent cells include, for example, antigen presenting cells such as Langerhans' cells and arboreal cells.

According to another aspect of the present invention, there is provided use of ATP or its pharmaceutically acceptable salt, its solvate, or its derivative having a physiological function for the production of an immunoadjuvant. Further, according to still another aspect of the present invention, there is provided use of a combination of ATP or its pharmaceutically acceptable salt, its solvate, or its derivative having a physiological function with an antigenic substance which can induce the production of an antibody having immunosuppressive activity for the production of a therapeutic or preventive agent for transplant rejection in organisms.

Organsisms in the present invention are preferably mammals. More preferred are humans, cattle or cows, pigs, horses, sheeps, dogs or cats. Humans are still more preferred.

### EXAMPLES

The present invention is further illustrated by the following Examples that are not intended as a limitation of the invention.

### Test Example 1

### Confirmation of Increased Production Amount of Antibody by Immunoadjuvant

The following test was carried out according to the following procedure to confirm the amount of antibody produced upon the administration of ATP together with an antigenic substance. The case where only an antigenic substance had been administered and the case where an antigenic substance and a cholera toxin as a mucosal immunoadjuvant had been administered, were selected as a reference example in the test.

### Preparation of Antigenic Substance and Immunoadjuvant

A mixture of a peptide having an amino acid sequence represented by SEQ ID No. 1 and a complex of the peptide with KLH was used as an antigenic substance.

In the preparation of the antigenic substance, the peptide having an amino acid sequence represented by SEQ ID No. 1 was first synthesized by an Fmoc peptide solid phase synthesis method (production apparatus; ABI430 manufactured by Applied Biosystems Inc.). Further, the complex of the peptide with KLH (manufactured by Sigma-Aldrich Co.) was synthesized by stirring a solution of 5 mg of the peptide, about 20 mg of KLH, and 30 μg of glutaraldehyde (manufactured by Katayama Chemical Industry Corp.) in a phosphate buffer (pH 8.0) at room temperature for about 6 hr.

Next, 10 μg of the peptide and 10 μg of the complex were mixed together in PBS to give an antigenic substance (10 μg peptide, 10 μg complex/0.2 mL PBS).

ATP (manufactured by Sigma-Aldrich Co.) was provided as an immunoadjuvant.

Cholera toxin (manufactured by Sigma-Aldrich Co.) was used as an adjuvant for a reference example.

The antigenic substance and the immunoadjuvant were used in a tape preparation form in the following test according to the following procedure.

At the outset, the antigenic substance (20 μg), ATP (20 mg), and a water soluble ointment base (a mixture of Macrogol 4000: Macrogol 1500: propylene glycol=3:1:1 wherein Macrogol 1500 is an equiamount mixture of Macrogol 1540 with Macrogol 300) were mixed together. The mixture (100 mg) was then coated on a tape for a patch test (an adhesive plaster for a patch test, tradename: Torii) to give a tape preparation.

Immunization

The antigenic substance (10 μg peptide, 10 μg complex/0.1 mL PBS) was intraperitoneally administered to Balb/c mice (female, 4-week old, n=4, manufactured by ORIENTAL YEAST Co., Ltd.).

When two weeks and four weeks had elapsed after the intraperitoneal administration, the above tape preparation was applied to the mice and was maintained in this state for 72 hr to dermal administer the antigenic substance and the immunoadjuvant. In this case, the tape preparation application site was previously subjected to hair shaving and full dehairing with a depilatory cream (tradename: Epilat, manufactured by Kanebo Ltd.). Further, the skin was dried for 1 to 2 hr, and the deadskin was then removed by tape stripping.

A blood sample was collected from each of the mice at the time of antigenic substance administration, about one week after the tape preparation application, and 25 days after the start of the test.

Measurement of production amount of antibody by ELISA

A blood sample was collected from each mouse, and the amount of the antibody in each mouse serum was then determined by ELISA according to the following procedure. In the following description, OVA-SSV is a complex of ovalbumin with a peptide having an amino acid sequence represented by SEQ ID No. 1. OVA-SSV was synthesized in the same manner as in the production of the complex of the peptide with KLH.

At the outset, a histone H1 solution (20 μg/mL, manufactured by Roche) or an OVA-SSV solution (OVA-SSV: 0.387 mg/mL, solvent: 0.02 M phosphate buffer, 0.9% NaCl, pH 8.0) were prepared using a 0.1 M NaHCO₃ (pH 9.3) solution. Next, the resultant solution was added 50 μL by 50 μL in each well of a 96-hole plate. The mixture was allowed to stand at room temperature for one hr. Each well was then washed three times with PBST. Thereafter, 150 μL of a PBS solution (3% milk, PBS solution containing 1% BSA) was added to each well, and the mixture was incubated at 37° C. for one hr. Each well was then washed three times with PBST, and 50 μL of a mouse serum diluted with PBST by a factor of 1000 was added thereto. The wells were then allowed to stand at room temperature for one hr. Each well was then washed three times with PBST. 50 μL of a peroxidase labelled mouse IgG (manufactured by Sigma-Aldrich Co.) which had been diluted with PBST by a factor of 2000 to 4000 was added to the wells. The wells were then allowed to stand at room temperature for one hr. Next, each well was washed three times with PBST, and ABTS (2,2′-azino-bis[3-ethylbenzoline-6-sulfonate], manufactured by Sigma-Aldrich Co.) was added as a chromophoric substrate, and incubation was then carried out for 30 to 60 min. Thereafter, the absorbance of each well was measured with Multiscan Ascent (manufactured by Thermo Labsystems, wavelength 405 nm).

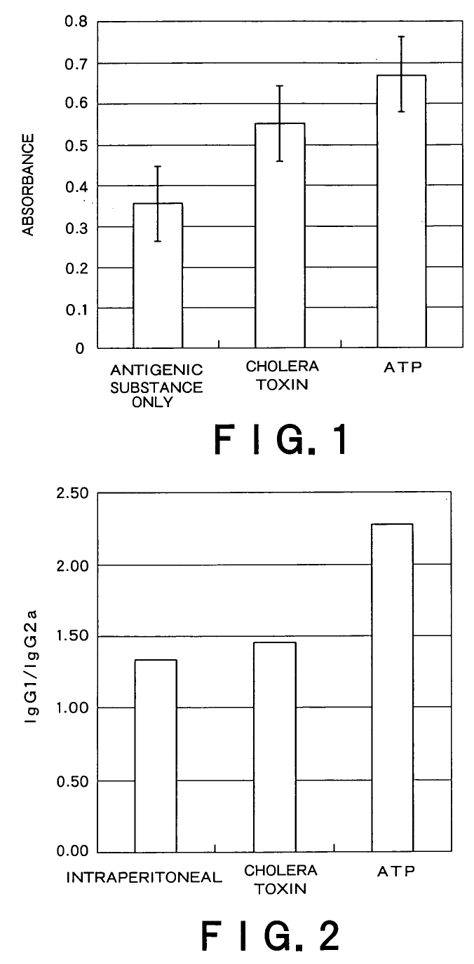

As a result, the average±standard error of the absorbance for the serum sample on the 25th day from the start of the test in each group was as shown in FIG. 1.

The average±standard error of the absorbance of the measured sample was 0.670±0.033 in the case of dermal administration of the antigenic substance together with ATP, was 0.355±0.062 in the case of the dermal administration of only the antigenic substance, and was 0.551±0.202 in the case of dermal administration of the antigenic substance together with cholera toxin. It was found that the production amount of the antibody with the use of the ATP as an immunoadjuvant was larger than that in the case where only the antigenic substance was administered, or in the case where the cholera toxin was used as an immunoajudvant.

### Test Example 2

### Measurement of IgG1/IgG2 Ratio

The blood sample collected on the 25th day from the start of the test was provided, and the IgG1/IgG2 ratio was measured as an index of Th2/Th1 balance.

Further, a blood sample obtained by inoculating only the antigenic substance by intraperitoneal administration instead of the dermal administration at the same time as in the administration schedule in Test Example 1 was used as a control blood sample for comparison.

The IgG1/IgG2 ratio was measured with a Mouse Monoclonal Antibody Isotyping Reagents kit (SIGMA).

Specifically, a mouse serum was diluted with PBS by a factor of 1000 to give a solution. The solution (100 μL) was added to wells in a plate and was incubated at 37° C. for one hr. Next, each well was washed three times with PBS, and 100 μL of isotyping specific reagents (reagents containing IgA, IgG1, IgG2a, and IgG2b), which had been diluted by a factor of 1000, were added to the wells followed by incubation at room temperature for 30 min. Each well was washed three times with PBST. A peroxidase labelled mouse IgG (manufactured by SIGMA) (100 μL), which had been diluted with PBST by a factor of 5000, was added to the wells, and the wells were allowed to stand at room temperature for one hr. The wells were then washed three times with PBST. Thereafter, ABTS was added as a chromophoric substrate, and incubation was carried out for 5 to 10 min. For each well, the absorbance was measured with Multiscan Ascent (manufactured by Thermo Labsystems, wave length 405 nm).

Thereafter, the absorbance of measuring samples in each group was as shown in FIG. 2.

The average of the absorbance of the measured sample was 2.28 in the case of dermal administration of the antigenic substance together with ATP, was 1.33 in the case of the intraperitoneal administration of only the antigenic substance, and was 1.46 in the case of dermal administration of the antigenic substance together with cholera toxin. When the ATP was used as an immunoadjuvant, it was found that the IgG1/IgG2 ratio was larger than that in the case where only the antigenic substance was intraperitoneally administered, or in the case where the cholera toxin was used as an immunoadjuvant. It was confirmed from the data

on the IgG1/IgG2 ratio that, when ATP is used as an immunoadjuvant, the humoral immunity is induced in such a state that the Th2 cells are predominant as compared with the case where only the antigenic substance is intraperitoneally administered or the case where cholera toxin was used as the immunoadjuvant.

1. An immunoadjuvant comprising ATP or its pharmaceutically acceptable salt, its solvate, or its derivative having a physiological function.

2. The immunoadjuvant according to claim 1, wherein the derivative is an ester or an amide.

3. The immunoadjuvant according to claim 1, which comprises ATP or its pharmaceutically acceptable salt or its solvate.

4. The immunoadjuvant according to claim 1, for dermal administration.

5. A vaccine composition comprising an immunoadjuvant according to claim 1 and an antigenic substance.

6. The vaccine composition according to claim 5, wherein the antigenic substance is selected from the group consisting of virus-derived antigens, bacteria-derived antigens, cancer-related antigens, and combinations thereof.

7. The vaccine composition according to claim 5, wherein the antigenic substance is selected from the group consisting of peptides, proteins, carbohydrates, lipids, nucleic acids, toxoids, and combinations thereof.

8. The vaccine composition according to claim 7, wherein the antigenic substance is a peptide or a protein.

9. The vaccine composition according to claim 5, wherein the antigenic substance comprises a peptide selected from the following peptides (a) and (b):
(a) a peptide having an amino acid sequence represented by SEQ ID No. 1; and
(b) a polypeptide comprising an amino acid sequence represented by SEQ ID No. 1 wherein one or a few amino acids have been substituted, deleted, or added, the polypeptide being functionally equivalent to the peptide described in the item (a).

10. The vaccine composition according to claim 5, which further comprises a pharmaceutically acceptable carrier.

11. The vaccine composition according to claim 10, wherein the carrier is bound to the antigenic substance.

12. The vaccine composition according to claim 10, wherein the carrier is keyhole limpet hemocyanine, ovalbumin, or bovine serum albumin.

13. The vaccine composition according to claim 12, wherein the carrier is keyhole limpet hemocyanine.

14. The vaccine composition according to claim 5, which further comprises a component selected from the group consisting of superantigens, cytokines, cholera toxins and mutants thereof, heat-labile enterotoxins and mutants thereof, and CpG oligonucleotides.

15. The vaccine composition according to claim 9, for use in the treatment or prevention of transplant rejection in organisms.

16. The vaccine composition according to claim 5, for use in pharmaceutical preparations.

17. The vaccine composition according to claim 16, which is in the form of a transdermal absorption preparation.

18. A method for increasing the amount of an antibody produced against an antigenic substance in an organism, the method comprising administering an immunologically effective amount of the antigenic substance, and ATP or its pharmaceutically acceptable salt, its solvate, or its derivative having a physiological function in an amount effective as an immunoadjuvant simultaneously or successively into the organism.

19. A method for inhibiting transplant rejection in organisms, the method comprising administering an immunologically effective amount of an antigenic substance according to claim 9, and ATP or its pharmaceutically acceptable salt, its solvate, or its derivative having a physiological function in an amount effective as an immunoadjuvant simultaneously or successively into the organism.
